(19) United States
(12) Patent Application Publication
(10) Pub. No.: US 2006/0150261 A1
(43) Pub. Date: Jul. 6, 2006

(54) Intracellular antibodies for a retrovirus protein

(75) Inventors: Dubravka Drabek, Rotterdam (NL); Sylvia Dekker, Delft (NL); Franklin G. Grosveld, Rotterdam (NL)

Correspondence Address: PALMER & DODGE, LLP;KATHLEEN M. WILLIAMS, 111 HUNTINGTON AVENUE, BOSTON, MA, 02199, US

(73) Assignee: Erasmus University

(21) Appl. No.: 11/372,250

(22) Filed: Mar. 9, 2006

Related U.S. Application Data
(63) Continuation of application No. 10/894,194, filed on Jul. 19, 2004, now abandoned.
Application No. 10/894,194 is a continuation of application No. PCT/IB03/00653, filed on Jan. 24, 2003.

(30) Foreign Application Priority Data
Jan. 24, 2002 (GB) 0201611.1

(51) Int. Cl. A01K 67/027

(52) U.S. Cl. 800/6; 800/14

(57) ABSTRACT

A transgenic organism is provided comprising a polynucleotide construct encoding an intracellular antibody which disrupts the catalysis of the production of the xenoantigen galactose α 1,3 galactose and/or a polynucleotide construct which encodes an intracellular antibody which binds specifically to a retrovirus protein, such as a PERV particle protein. Also described are methods for the production of such organisms. Cells, tissues and organs of the transgenic organism may be used in xenotransplantation.

### FIELD OF THE INVENTION

The present invention relates to methods of producing transgenic animals. In particular it relates to the use of polynucleotide constructs to enable intracellular expression of polypeptides whose effect is to enable the use of said animals in xenotransplantation whilst providing improved safety and tolerance.

### BACKGROUND TO THE INVENTION

Although great improvement in survival rates of patients undergoing transplantation have been achieved in recent years, success rates are limited by shortage of available donor organs and tissues. As a result, waiting list are getting longer and the number of patients dying while waiting for a transplant is increasing. In the USA, for example, a third of patients on organ waiting lists die before undergoing transplant surgery.

The shortage in available organs for allotransplantation has resulted in a search for suitable organs for xenotransplantation. Initially research focussed on animals of closely related species to humans, such as primates. However, the availability of primates is limited by a number of factors including the relatively small populations of suitable primate populations, the fact that many of these species are endangered, concerns over interspecies viral transmission, and ethical considerations. As a result, non-primate species have been considered as pots donors. A particularly preferred candidate as a xenotransplantation donor is the pig (Sus scrofa), which, as well as being easy to breed and handle, has organs of similar size to that of humans. The pig also has a lower risk of infectious disease in comparison to primates.

Immunological Rejection

The success of xenotransplantation between pigs and humans has been restricted due to severe immunological rejection of the grafted tissue, either by naturally occurring antibodies in the recipient or by rapid cell mediated rejection. This attack, which is known as hyperacute rejection involves preformed antibodies fixing complement, which leads to damage to the endothelial cell lining of blood vessels, resulting in haemorrhage and oedema with aggregation of platelets blocking the microvasculature, depriving the graft of its blood supply.

Complement Activation

In interspecies transplantation, the graft recipient's complement is primarily activated via the classical complement pathways, in which C1 binds to antibody. The C1 s subunit acquires esterase activity and cleaves C4 to generate activated C4b, surface bound forms of which act as a binding site for C2. The resultant C4b2a complex has “C3-convertase” activity and splits C3 to produce C3a, which has anaphylatoxin activity which contributes to complement-mediated damage, and C3b, which is membrane bound and can cause immune adherence of the antigen-antibody-C3b complex, so facilitating subsequent phagocytosis.

The final stage of complement activation is the formation of the membrane attack complex. C3b binds C5 which is then cleaved by C5 convertase, a trimolecular complex of C4b2a3b, to give C5a, a potent anaphylatoxin, and C5b, which binds C6, then C7 to form a complex which preferentially inserts into lipid bilayers. Finally C8 and C9 are recruited to generate the membrane attack complex (MAC).

The complement cascade is subject to regulation by a number of complement control proteins. For example, decay accelerating factor (DAF) and CR1 inhibit the binding of C2 to C4b and promote the dissociation of C2a from C4b with CR1 and Membrane co-factor protein (MCP) promoting the catabolism of C4b by Factor I. Further regulation is provided by membrane proteins which protect against lysis by the MAC. CD59 binds to C8 in C5b-8 complexes, inhibiting the insertion and unfolding of C9, with homologous restriction factor, HRF, known to have a similar function.

Strategies to Limit Immune Rejection

A number of approaches have been taken in attempts to limit hyperacute rejection by attempts to reduce the activation of complement. Some limited success in reducing complement activity has been demonstrated by the administration of large amounts of cobra venom factor (or soluble complement receptor prior to transplantation, with prolonged xenograft survival demonstrated (Platt et al., Immunol. Today 11:450-456 (1990); Lacer et al., Trans. Proc. 19:1153-1154 (1987)). In these studies, xenografts were maintained for days or weeks if the host complement was continuously suppressed.

An alternative approach has been the administration of complement regulatory proteins, or homologous complement restriction factors. WO 91/05855 describes the preparation of transgenic mice bearing a transgene encoding human membrane cofactor protein (MCP) (CD46) or human decay accelerating factor (DAF). Similarly, Yannoutsos et al., First Int'l Conqr. Xenotr., Abstracts, p7 (1991), describes the development of transgenic mice expressing human DAF and MCP. Transgenic mice and pigs which contain a human DAF gene have been produced using a partial genomic DNA fragment (Cary et al., Trans. Proc. 25:400-401, 1993: Cozzi et al., Trans. Proc. 27:319-320, 1995). Foder et al. (Proc. Nat. Acad. Sci. USA, 91: 11153-57 (1994)) sought to produce transgenic mice and swine producing the complement inhibitor CD59 by expressing CD59 under the control of the promoter of the MHC Class I gene H2K.sup.b, which encodes an antigen which is a predominant endothelial cell surface antigen

PCT/US93/08889 (WO 9105855) describes the expression of complement inhibitors in the red blood cells of transgenic animals, which then transfer the proteins to the vascular endothelium of their organs and tissues. However, this method requires routine reperfusion with the transgenic animal's blood in order to maintain high expression.

Galactose α-1,3-Galactose

An alternative approach to the modulation of the complement reaction to antigens on a donor tissue is the modulation of the xenoantigenicity of the donor organ itself. It is now believed that the major target of xenoreactive natural antibodies is the oligosaccharide epitope galactose α-1,3-galactose (“α-Gal”) (Sandrin et al., P.N.A.S. 5 90:11391-11395 (1993) and Transplantation Reviews, 8:134-149 (1994)). This epitope, which is absent in primates, Old World monkeys and humans (Good et al, Transplant Proc. 24:559-562 (1992) and Galili et al., P.N.A.S. 84:1369-1373 (1987)) is believed to be synthesised by the enzyme Galβ1,4GlcNAc3-α-D galactosyltransferase (or “α-1,3 galactosyltransferase”; EC 2.4.1.51) which catalyzes the addition of galactose to a N-acetyllactosamine (N-lac) core (Blanken et al., J. Biol. Chem. 260:12927-12934 (1985)).

U.S. Pat. No. 6,166,288 proposes the production of transgenic animals such as pigs (or mice) which express an enzyme such as α-1,2 fucosyltransferase to mask or reduce the level of xenoreactive antigens, such as Gal epitope, by competing with α-1,3 galactosyl transferase for its N-lac substrate in order to reduce the expression of the xenoantigenic galactose α-1,3-galactose.

U.S. Pat. No. 6,096,725 discloses the use of oligosaccharides containing a galactose α-1,3-galactose motif to competitively bind anti-galactose α-1,3-galactose antibodies in order to reduce the binding of such antibodies to the xenograft and help reduce or slow rejection.

Although prior art methods directed to limiting hyperacute rejection may reduce the immunological reaction to galactose α-1,3-galactose to some extent, the presence of even small amounts of the galactose α-1,3-galactose antigen on the surface of graft cells is potent enough to induce hyperacute rejection. Accordingly there is a need for improved methods of reducing gal antigen expression.

Endogenous Retroviruses

A further concern raised over interspecies transplantation is the possibility of cross species transmission of diseases and viruses.

While most of the known exogenous pathogens can be controlled by breeding under specified pathogen free (spf) conditions, viruses such as endogenous retroviruses cannot be eliminated easily.

The DNA of most investigated species comprise integrated endogenous retroviruses. Although many of such endogenous retroviruses are not believed to be harmful to their natural host species, they may nevertheless be harmful to other species. Therefore concerns remain regarding the possibility of cross-species infection. The presence of such retroviruses in the cells of donor organisms have led to concern over the safety of their use in xenotransplantation, given that some retroviruses are widely believed to have crossed the species barrier.

Porcine endogenous retroviruses (PERVs) are present in the genome of all pigs and are believed to be harmless to pigs. On replication in the infected cell, new PERV particles are produced and released from the cells, the RNA within the particle being enclosed by an envelope protein in part derived from the membrane of the cell. Although several studies have found no evidence of infection of human cells by PERVs in patients and controls who have received pig blood plasma or xenotransplants (Paradis et al, Science 285, 1236-1241 (1999), Heneine et al, Lancet 352, 695-699 (1998)), other studies have shown that PERVs can infect human cell lines in vitro and that transplantation of pig pancreatic islets into SCID mice resulted in ongoing viral expression and infection of the mice (van der Laan et al, Nature 407, 90-94 (2000)).

Recent studies have shown that there are approximately 50 proviral integration sites in the pig genome (1,2,3) of which a number are thought to occur at the same position in different pig breeds (H. Kuipers, unpubl.). This makes a knock out or breeding strategy for elimination of PERVs impossible.

At least two classes of porcine retroviruses (PERV-A and PERV-B) are able to infect human cells in vitro (3-6). Although long term infection after transplantation has not been found (7-9), PERVs are able to infect mouse cells in vivo (10,11). Hence there is a possibility that PERVs might lead to a malignant, immunosuppressive (12) or other diseases in the recipient of a porcine transplant and that they might spread beyond the recipient into the human population, all potential risks, that presently do not allow xenotransplantation (as described, for example, in reference 13).

Accordingly there is a need for a method of reducing risk of transmission of retroviruses such as PERVs.

Thus, there is a need for methods which reduce the immunological response of a recipient to a graft and methods which address the problem of the presence of PERVs in donor tissue.

### SUMMARY OF THE INVENTION

The present invention is based on the finding that intracellular expression of antibodies or antibody fragments by the introduction of a gene encoding such an antibody can improve the safety and tolerance of xenotransplants.

Thus, according to a first aspect of the present invention, there is provided a non-human transgenic animal comprising a polynucleotide construct which includes a nucleotide sequence encoding an intracellular immunoglobulin.

In one embodiment, the present invention is based on the surprising finding that expression of galactose α-1,3-galactose epitopes on the cell surface of an animal may be reduced or eliminated by the introduction of a gene encoding an antibody which binds specifically to an enzyme required for the production of the epitope.

In a preferred embodiment said immunoglobulin is able to bind specifically to an enzyme which catalyses the production of galactose α-1,3-galactose.

An immunoglobulin “binds specifically” to a protein, e.g. enzyme, polypeptide or peptide molecule if such binding is not competitively inhibited by the presence of a non-related molecule.

In a second aspect, the invention provides a method of producing a non-human transgenic organism, said method comprising inserting into the genome of said organism a polynucleotide construct which includes a nucleotide sequence encoding an intracellular immunoglobulin.

Suitably, said intracellular immunoglobulin is able to bind specifically to an enzyme which catalyses the production of galactose α-1,3-galactose.

By integrating into the genome of the transgenic organism a construct encoding an immunoglobulin such as an antibody with specificity for an enzyme which catalyses the production of galactose α-1,3-galactose, the synthesis of galactose α-1,3-galactose and its expression on the surface of cells of transgenic organisms may be reduced or prevented. Cells, tissues and organs of such organisms will therefore be less immunogenic and such animals may thus be of use as a source of tissues and/or organs for xenotransplantation.

Thus, in a third aspect, the invention extends to a method for preparing organs, tissues or cells for xenotransplantation comprising:

providing a transgenic organism according to the first aspect of the invention or produced according to the method of the second aspect of the invention, and

isolating said organ, tissue or cell from said transgenic organism.

In preferred embodiments of these aspects of the invention, the enzyme to which the intracellular antibody binds is α-1,3 galactosyl transferase.

The reduction or elimination of galactose α-1,3-galactose epitopes from cells or tissues of transgenic animals reduces the immunogenicity of the cells or tissues and thus may be useful in reducing or preventing graft rejection when such cells or tissues are transplanted into a recipient organism.

However, as described above, in animals infected with a retrovirus such as a porcine endogenous retrovirus (PERV), replication of the retrovirus within the host cell results in release of new retroviral particles from the host cell, the envelope protein of the retrovirus particles being part derived from the cell membrane of the host cell.

Therefore, in such animals in which galactose α-1,3-galactose is expressed on the surface of cells, the retrovirus particles, e.g. PERV particles, released from such cells also express the oligosaccharide on the envelope protein of the particle.

By eliminating the expression of galactose α-1,3-galactose from the surface of cells of an orgasm, the expression of galactose α-1,3-galactose on the surface of retroviral particles such as PERVs may also be eliminated, reducing or eliminating recognition of the retroviral particles by host anti-galactose α-1,3-galactose antibodies (31). The same is true for pigs transgenic for human CD46, CD55 or CD59. This lack of recipient anti-galactose α-1,3-galactose response to the retrovirus may promote retroviral infection of the host through escaping the complement-mediated virolysis (32).

The present inventors have found that this problem may be obviated by introduction of a gene encoding an intracellular immunoglobulin able to bind specifically to a retrovirus protein into the genome of a host organism. By interfering with the packaging and/or formation of endogenous retroviral particles from the cells of transplanted tissues, the release of retroviruses such as PERVs from the cells and tissues of the organism may be reduced or prevented completely.

Accordingly, in a further aspect of the invention, the present invention provides a method of disrupting release of a retroviral particle from a cell of a host organism, comprising inserting into the genome of the organism a polynucleotide construct encoding an intracellular immunoglobulin which binds specifically to a retrovirus protein.

In the context of the present invention, a retrovirus protein is any protein or part thereof which, upon binding by an immunoglobulin, is inhibited from forming, packaging or releasing a retrovirus particle from a host cell.

Retroviruses consist of the gag, pol and env genes flanked by LTRs (long terminal repeats). The gag gene encodes the structural virion proteins. The pol gene encodes reverse transcriptase (RT), which ensures synthesis of DNA complementary to viral RNA, and in addition the pol gene encodes protease and integrase which are required for proviral integration. The env gene encodes two envelope proteins denoted transmembrane protein (TM), and surface unit (SU). These two proteins mediate the interactions with host cell membranes and make exit from host cells and entry into cells possible by interaction with receptors at the cell membrane.

While C-type PERVs differ significantly in the env gene encoding the envelope proteins, the genes for the polymerase (pol) and the group sic antigens (gag) show high homology between all potentially xenotropic classes of PERVs (6).

Accordingly in a preferred embodiment of the invention, the immunoglobulin binds specifically to a retrovirus protein selected from Gag or Pol. As demonstrated herein, anti-sera raised against Gag or Pol, will react to both xenotropic/polytropic classes of PERVs (PERV-A and PERV-B) as well as the ecotropic PERV-C class.

The invention also provides a non-human transgenic animal comprising a polynucleotide construct which includes a nucleotide sequence encoding an intracellular immunoglobulin able to bind specifically to a retrovirus protein.

In preferred embodiments of the invention the retrovirus protein to which the intracellular immunoglobulin or antibody is capable of specifically binding is a retroviral GAG protein or an ENV protein. In particular, the immunoglobulins are immunoglobulins which selectively bind the p10, p12, p15 or p30 nucleocapsid portion, or p27, of the GAG polyprotein.

In a particularly preferred embodiment, the intracellular immunoglobulin is a single domain antibody (VHH), preferably a single domain antibody derived from Lama glama.

Suitably, said intracellular immunoglobulin has an amino acid sequence as set out in FIG. 2. In a particularly preferred embodiment, the intracellular immunoglobulin is a single domain antibody (VHH) against p15, the matrix domain protein of the PERV Gag polyprotein.

Preferably said non-human transgenic animal further comprises a nucleotide sequence encoding an intracellular immunoglobulin able to bind specifically to an enzyme which catalyses the production of galactose α-1,3-galactose.

Also provided is a method of producing a non-human transgenic organism said method comprising inserting into the genome of said organism a polynucleotide construct which includes a nucleotide sequence encoding an intracellular immunoglobulin able to bind specifically to a retrovirus protein.

In one embodiment the method further comprises inserting into the genome of said organism a polynucleotide construct which includes a nucleotide sequence encoding an intracellular immunoglobulin able to bind specifically to an enzyme which catalyses the production of galactose α-1,3-galactose. Transgenic organisms comprising both nucleotide sequences are also encompassed by the invention.

In a preferred embodiment of the invention, the retroviral particle is porcine endogenous retroviral particle (PERV).

The invention thus further provides a method of producing a transgenic pig with reduced expression of PERV particles, said method comprising inserting into the genome of said organism a polynucleotide construct which includes a nucleotide sequence encoding an intracellular immunoglobulin able to bind specifically to a PERV protein.

Transgenic pigs produced using a method according to the invention are also provided.

The invention further extends to polynucleotide constructs suitable for use in the methods of the invention and immunoglobulins encoded by such polynucleotide constructs. Suitably said polynucleotide constructs are selected from polynucleotides encoding Gag positive binders having the amino acid sequences as set out in FIG. 2. In a particularly preferred embodiment the polynucleotide construct encodes the amino acid sequence which encodes any of the single domain antibodies A5, E11 or D2.

Polynucleotide constructs may her comprise a nucleic acid sequence encoding at least one complement inhibiting protein, such as CD59, DAF and MCP. Expression of the construct within a host cell may thus result in production of one or more complement inhibitors to reduce the activation of complement which may be induced by xenoantigens other than galactose α-1,3-galactose or indeed by any residual galactose α-1,3-galactose.

In another embodiment there is provided an isolated polypeptide having the amino acid sequence of any of the gag positive binders set out in FIG. 2, or variant, derivatives or fragments thereof. In a particularly preferred embodiment, the isolated polypeptide encodes any of the single domain antibodies A5, E11 or D2.

The gag positive binders can also be used in a method of diagnosing the presence of PERVs. Accordingly, in another aspect of the invention there is provided a method of detecting the presence of a PERV in a sample, said method comprising taking said sample and incubating in the presence of a gag positive binder in accordance with the invention. Suitably said gag positive binder is linked to a detectable moiety to enable binding to be detected. Methods for determining immunoglobulin binding to a sample are well known to those skilled in the art and include western blotting, immunofluoresence and so forth.

### BRIEF DESCRIPTION OF THE FIGURES

FIG. 1 Lama glama antiserum recognizes Gag and contains several VHHs with different affinities for vial Gag. A. PERV's detection in PK15 cells cryosections by immunoelectron microscopy using llama α Gag antiserum. Arrows indicate viral particles. B. Western blot showing specificity for the 60 kD Gag polyprotein and or different Gag domains. A4, A5, E11, C1, B10 and H2 are antibodies against matrix protein p15 while D2 and G12 bind to capsid protein p27. All VHHs recognize whole Gag. C. BIAcore affinity measurements. Equal amounts of soluble reactive VHHs from periplasmic fractions were used to measure relative binding to immobilised Gag protein on a BIAcore sensorchip.

FIG. 1D shows that the epitope of the gag protein p15 (133 amino acids in size) that is recognised by the A5 antibody maps between amino acid 47 and 113 and involves the sequence PPPWV.

FIG. 2 Amino acid sequences of Gag positive binders and constructs used to intracellularly express single domain antibody A5. A. Alignment of 8 different llama antibodies against the PERV-B Gag protein. The VHH structural elements (CDRs and FRs), hinge region and CH2 exon are indicated. B. The vectors used for transfection experiments in PK15 cells. Left: Tet-on regulatory plasmid pUHrT 62-1-puro. Right: response plasmid 2×p(A)BiDi-A5-Myc containing A5 VHH in frame with the Myc-tag cloned on one side of the bidirectional Tre responsive promoter (Clontech) and the neomycin resistance gene.

FIG. 3. Immunofluorescent staining showing doxycycline induced production of A5 VHH and its influence on Gag expression. PK15 cells were stably transfected with the tet-on regulatory plasmid, so called tet-on line (panel A) and two clones 17 (panel B) and 13 (panel C) of tet-on line, which were additionally stably transfected with the response plasmid containing A5 VHH. The cells were either not treated (A1, B1, C1) or treated for 48 hours with doxycycline (A2, B2, C2). A3, B3, C3: same fields as A2, B2 and C2 of dox induced cells showing Gag expression in green and A5 VHH expression in red.

FIG. 4. Viral production (PERVA/B) by PK15 cells is blocked upon expression of A5 VHH. A. Relative RT activity in the cell free supernatant. Light blue bars represent RT activity in non-treated samples; dark blue bars represent RT activity in supernatants from dox treated cells. The decrease in RT activity is proportional to the number of A5 VHH expressing cells. B. Western blot showing gag expression in cell lysates upon dox induction in the different clones. Within each clone β tubulin is stained as a loading control. C. RT-PCR of serially diluted viral cDNA preparations. 2, 0.4 and 0.08 refer to μl of template used in reaction. The gel was stained with Ethidium Bromide. Both PERV-A and PERV-B are blocked (>25 fold decrease) by the expression of A5VHH.

FIG. 5 shows two clones E11-1 and E11-15 made by transfecting the E11 single chain antibody into PK15 Tet-on (PERV producing cell line). Induction of expression of the antibody (red stain) results in a loss of virus production (green speckles), although the gag protein is still visible in the cells (diffuse green staining). E11 recognises epitopes on the PERV A and B gag protein p15 different from those recognised by antibody A5.

FIG. 6 shows two clones D2-1 and D2-2 made by transfecting the D2 single chain antibody into PK15 Tet-on (PERV producing cell line). Induction of expression of the antibody (red stain) results in a loss of virus production (green speckles), although the gag protein is still visible in the cells (diffuse green staining). D2 recognises the p30 gag protein of PERV A and B.

### DETAILED DESCRIPTION

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2nd ed. (1989) Cold Spring Harbor Laboratory Press, Cold Spring Harbor, N.Y. and Ausbel et al., Short Protocols in Molecular Biology (1999) 4th ed., John Wiley & Sons, Inc.—and the fill version entitled Current Protocols in Molecular Biology, which are incorporated herein by reference) and chemical methods.

Immunoglobulins

Immunoglobulins, in the context of the present invention, refer to any moieties which are capable of binding specifically to an enzyme which catalyses the production of galactose α-1,3-galactose or which are capable of binding specifically to a retrovirus protein, preferably a PERV protein.

In particular, they include members of the immunoglobulin superfamily, a family of polypeptides which comprise the immunoglobulin fold characteristic of antibody molecules, which contains two β sheets and, usually, a conserved disulphide bond. The present invention is applicable to all immunoglobulin superfamily molecules which are capable of binding specifically to an enzyme which catalyses the production of galactose α-1,3-galactose or which are capable of binding specifically to a retrovirus protein. Preferably, the present invention relates to antibodies.

Antibodies, as used herein, refers to complete antibodies or antibody fragments capable of binding to a an enzyme which catalyses the production of galactose α-1,3-galactose, and include Fv, ScFv, Fab′ and F(ab′)₂, monoclonal and polyclonal antibodies, engineered antibodies including chimeric, CDR-grafted (complementarity determining region-grafted) and humanised antibodies, diabodies, and artificially selected antibodies produced using phage display or alternative techniques. Preferably, the anybody is a single chain antibody, such as a heavy chain only antibody or a camelid single domain VHH antibody. The invention may employ CDR-grafted antibodies, which are preferably CDR-grafted light chain and heavy chain variable domains only. Advantageously, the heavy chain variable domain and the light chain variable domain are linked by way of a spacer group, optionally coping a signal sequence facilitating the processing of the antibody in the host cell and/or a DNA coding for a peptide facilitating the purification of the antibody and/or a cleavage site and/or a peptide spacer and/or an effector molecule. Such antibodies are known as scFvs.

Diabodies are multimers of polypeptides, each polypeptide comprising first and second domains comprising a binding region of an immunoglobulin light chain and a binding region of an immunoglobulin heavy chain respectively, the two domains being linked (e.g. by a peptide linker) but unable to associate with each other to form an antigen binding site. Antigen binding sites are formed by the association of the first domain of one polypeptide with the second domain of another polypeptide within the multimer. (WO94/13804).

It may be preferable to use diabodies or ScFv dimers rather than whole antibodies as diabodies and scFv dimers can be constructed without an Fc region, using only variable domains, potentially reducing the effects of anti-idiotypic reaction.

As antibodies may be modified in a number of ways, the term “antibody” should be understood to refer to any specific binding member or substance having a binding domain with the require specificity. The antibodies for use in the invention may be altered antibodies comprising an effector protein such as a toxin or a label. Especially preferred are labels which allow the imaging of the distribution of the antibody in vivo. Such labels may be radioactive labels or radioopaque labels, such as metal particles, which are readily visualisable within the body of an organism, e.g. a transgenic organism or a patient into whom a tissue or organ has been transplanted. Moreover, they may be fluorescent labels or other labels which are visualisable on tissue samples removed from patients. Effector groups may be added prior to the selection of the antibodies or afterwards. Effector groups e.g. toxins may be used which denature an enzyme such as α-1,3 galactosyl transferase involved in the catalysis of the production of galactose α-1,3-galactose.

Intracellular Antibodies

In preferred embodiments of the invention, the immunoglobulins are intracellular immunoglobulins, preferably intracellular antibodies. “Intracellular” means inside a cell, and “intracellular immunoglobulins/antibodies” are immunoglobulins/antibodies which will bind to targets selectively within a cell. In normal circumstances, the biosynthesis of immunoglobulin occurs into the endoplasmic reticulum for secretion as antibody. However, antibodies may be expressed in the cell cytoplasm. Such intracellular antibodies or intrabodies have been demonstrated to function in antigen recognition in the cells of higher organisms (reviewed in Cattaneo, A. & Biocca, S. (1997) Intracellular Antibodies: Development and Applications Landes and Springer-Verlag). This interaction can influence the function of cellular proteins which have been successfully inhibited in the cytoplasm, the nucleus or in the secretory pathway.

This efficacy has been demonstrated for viral resistance in plant biotechnology (Tavladoraki, P., et al. (1993) Nature 366: 469-472) and several applications have been reported of intracellular antibodies binding to HIV viral proteins (Mhashilkar, A. M., et al. (1995) EMBO J 14: 1542-51; Duan, L. & Pomerantz, R. J. (1994) Nucleic Acids Res 22: 5433-8; Maciejewski, J. P., et al. (1995) Nat Med 1: 667-73; Levy-Mintz, P., et al. (1996) 1 Virol. 70: 8821-8832) and to oncogene products (Biocca, S., Pierandrei-Amaldi, P. & Cattaneo, A. (1993) Biochem Biophys Res Commun 197: 422-7; Biocca, S., Pierandrei-Amaldi, P., Campioni, N. & Cattaneo, A. (1994) Biotechnology (NY) 12: 396-9; Cochet, O., et al. (1998) Cancer Res 58: 1170-6). Antibodies which may function intracellularly may be identified using any technique known to the skilled person. Details of a preferred method are described infra.

In a particularly preferred embodiment of the invention, the immunoglobulins are camelid heavy chain only single chain antibodies. Camelids (camels and llamas) contain, in addition to normal heavy and light chain antibodies (2 light chains and 2 heavy chains in one antibody), single chain antibodies (containing only heavy chains). The latter are coded for by a distinct set of V_{H} segments referred to as V_{HH} genes.

Natural V_{HH} containing antibodies are missing the entire C_{H}1 domain of the constant region of the heavy chain. The exon coding for the C_{H}1 domain is present in the genome but is spliced out due to the loss of a functional splice acceptor sequence at the 5′side of the C_{H}1 exon. As a result the VDJ region is spliced onto the C_{H}2 exon. When a V_{HH} is recombined onto such constant regions (C_{γ}2, C_{γ}3) an antibody is produced that acts as a single chain antibody (i.e. an antibody of two heavy chains without a light chain interaction). Binding of an antigen is different from that seen with a conventional antibody, but high affinity is achieved the same way, i.e. through hypermutation of the variable region and selection of the cells expressing such high affinity antibodies. The same process of hypermutation seen in camelids may be replicated in transgenic animals.

Such single chain antibodies to a specific antigen may be obtained by screening an artificially constructed synthetic library of single chain antibodies or a library made from antibodies derived from immunised llamas or camels.

Alternatively, single chain antibodies to a specific antigen may be obtained by screening a library made from antibodies derived from immunised transgenic animals such as those described in co-pending GB patent application GB0110029.6. Camelid single chain antibodies which may be produced by said transgenic animals and which may be used in the present invention may be encoded by a camelid heavy chain immunoglobin locus comprising (i) a first locus including a V_{HH} region, a D region and a J region capable of recombining to form a VDJ coding sequence encoding a variable heavy chain polypeptide including a complete antigen binding site; and (ii) a second locus including at least one exon encoding a constant heavy chain polypeptide. The V_{HH} region of the first locus preferably comprises at least one V_{HH} exon. The V_{HH} exon may be a naturally occurring V_{HH} coding sequence such as found in camelids or a derivative of a naturally occurring V_{HH} coding sequence that includes any substitution of; variation of, modification of replacement of; deletion of or addition of one (or more) nucleic acids from or to the sequence of a naturally occurring V_{HH} coding sequence, provided the resultant nucleotide sequence is able to form a VDJ exon encoding a functional variable heavy chain polypeptide.

Naturally occurring V_{HH} exons may be obtained from a variety of sources readily apparent to one skilled in the art including camelid genomic DNA, cDNA, PAC, BAC or YAC libraries. Alternatively, the V_{HH} exons may be chemically synthesised using established techniques and the available nucleic acid sequence information. Preferably, the D region and J region of the first locus comprise at least one D exon and J exon respectively. The D exon and J exon may correspond to naturally occurring sequences. Preferably, the exon included in the second locus is one or more exons selected from the group of exons encoding a constant heavy chain (“C_{H} exons”) comprising: Cμ, Cδ, Cγ₁₋₄, Cε and Cα₁₋₂. The particular choice of D exon, J exon, and C_{H} exon, in particular the species of origin, will depend at least partly on the intended use of the construct. In the case of llamas, single chain antibodies are encoded from Cγ exons only. The other exons (including those of other species such as humans) may be engineered to allow use in single chain antibodies by deletion of the C_{H}1 exon. For example, where the construct is to be used to produce a transgenic animal, the choice of the exons may depend upon the intended use of antibodies produced therefrom. In the context of the present invention the D exon, J exon and C_{H} exon may originate from the same species as the transgenic animal in which the transgene is to be incorporated, such as a pig. Alternatively, the D exon, J exon and C_{H} exon may originate from the same species into which a transplant is to be introduced.

Further details of camelid antibodies and transgenic animals capable of producing such antibodies are described in co-pending GB patent application 0110029.6, the contents of which are herein incorporated by reference.

Production of Antibodies

General techniques for the production of antibodies discussed in, for example, Kohler and Milstein (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436S97, which are incorporated herein by reference.

In preferred embodiments of the invention, recombinant nucleic acids comprising an insert coding for a heavy chain variable domain and/or for a light chain variable domain of antibodies are employed. By definition such nucleic acids comprise coding single stranded nucleic acids, double stranded nucleic acids consisting of said coding nucleic acids and of complementary nucleic acids thereto, or these complementary (single stranded) nucleic acids themselves.

Furthermore, nucleic acids encoding a heavy chain variable domain and/or a light chain variable domain of antibodies can be enzymatically or chemically synthesised nucleic acids having the authentic sequence coding for a naturally-occurring heavy chain variable domain and/or for the light chain variable domain, or a mutant thereof. A mutant of the authentic sequence is a nucleic acid encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant nucleic acid is also intended to be a silent mutant wherein one or more nucleotides are replaced by other nucleotides with the new codons coding for the same amino acid(s). Such a mutant sequence is also a degenerated sequence. Degenerated sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly yeast, bacterial or mammalian cells, to obtain an optimal expression of the heavy chain variable domain and/or a light chain variable domain.

The term mutant is intended to include a DNA mutant obtained by in vitro or in vivo mutagenesis of DNA according to methods known in the art.

Recombinant DNA technology may be used to improve the antibodies of the invention. Thus, chimeric antibodies may be constructed in order to decrease the immunogenicity thereof in diagnostic or therapeutic applications. Moreover, immunogenicity within, for example, a transgenic organism such as a pig, may be minimised, by altering the antibodies by CDR grafting in a technique analogous to humanising antibodies [see European Patent Application 0,239,400 (Winter)] and, optionally, framework modification [see international patent application WO 90/07861 (Protein Design Labs)].

The invention therefore may also employ recombinant nucleic acids comprising an insert coding for a heavy chain variable domain of an antibody fused to a porcine constant domain. Likewise the invention concerns recombinant DNAs comprising an insert coding for a light chain variable domain of an antibody fused to a porcine constant domain κ or λ. Similarly, in order to reduce immunogenicity within a transplant recipient, the invention may employ recombinant nucleic acids comprising an insert coding for a heavy chain variable domain of an antibody fused to a human constant domain. Likewise the invention concerns recombinant DNAs comprising an insert coding for a light chain variable domain of an antibody fused to a human constant domain κ or λ.

As described above, single chain antibodies may also be engineered by deletion of C_{H}1 exons of a human antibody.

Antibodies may moreover be generated by mutagenesis of antibody genes to produce 5 artificial repertoires of antibodies. This technique allows the preparation of antibody libraries. As discussed further below; antibody libraries are also available commercially. Hence, the present invention advantageously employs artificial repertoires of immunoglobulins, preferably artificial ScFv repertoires, as an immunoglobulin source.

Selection of Immunoglobulins

Libraries and Selection Systems

Immunoglobulins which are able to bind specifically to an enzyme which catalyses the production of galactose α-1,3-galactose and thus may be used in the methods of the invention may be identified using any technique known to the skilled person. Such immunoglobulins may be conveniently isolated from libraries comprising artificial repertoires of immunoglobulin polypeptides. A “repertoire” refers to a set of molecules generated by random, semi-random or directed variation of one or more template molecules, at the nucleic acid level, in order to provide a multiplicity of binding specificities. Methods for generating repertoires are well characterised in the art.

Any library selection system may be used in conjunction with the invention. Selection protocols for isolating desired members of large libraries are known in the art, as typified by phage display techniques. Such systems, in which diverse peptide sequences are displayed on the surface of filamentous bacteriophage, have proven useful for creating loonies of antibody fragments (and the nucleotide sequences that encode them) for the in vitro selection and amplification of specific antibody fragments that bind a target antigen. The nucleotide sequences encoding the V_{H} and V_{L} regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected hazy member in bacterial cells. Furthermore, since the nucleotide sequence that encodes the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) Nature 348 552-554, Kang et al. (1991) Proc. Nat. A cad. Sci U.S.A., 88: 4363; Clackson et al. (1991) Nature, 352: 624; Lowman et al. (1991) Biochemistry, 30: 10832; Burton et al (1991) Proc. Nat. Acad Sci USA.; 88: 10134; Hoogenboom et al. (1991) Nucleic Acids Res., 19: 4133; Chang et al. (1991) J Immunol., 147: 3610; Breitling et al. (1991) Gene, 104: 147; Marks et al. (1991) J. Mol. Biol. 222, 581-597; Barbas et al. (1992) Proc. NatI. Acad. Sci. USA 89, 10164-10168; Hawkins and Winter (1992) .J Immunol., 22: 867; Marks et al., 1992, J. Biol. Chem., 267: 16007; Lerner et al. (1992) 20 Science, 258: 1313, incorporated herein by reference).

One particularly advantageous approach has been the use of scFv phage-libraries (Huston et al, 1988, Proc. Natl. Acad. Sci U.S.A, 85: 5879-5883; Chaudhary et al. (1990) Proc. Natl. Acad. Sci U.S.A., 87: 1066-1070; McCafferty et al. (1990) supra; Clackson et al. (1991) Nature 352, 624-628; Marks et al. (1991) supra; Chiswell et al (1992) Trends Biotech., 10: 80; Marks et al. (1992) supra). Various embodiments of scFv libraries displayed on bacteriophage coat proteins have been described.

Refinements of phage display approaches are also known, for example as described in WO96/06213, WO92/01047 (Medical Research Council et al.) and WO97/08320 (Morphosys), which are incorporated herein by reference.

Alternative library selection technologies include bacteriophage lambda expression systems, which may be screened directly as bacteriophage plaques or as colonies of lysogens, both as previously described (Huse et al. (1989) Science, 246: 1275; Caton and Koprowski (1990) Proc Natl. Acad. Sci. USA., 87; Mullinax et al. (1990) Proc. Nat. Acad Sci. USA., 87: 8095; Persson et al. (1991) Proc. Nat. Acad Sci. USA., 88: 2432) and are of use in the invention. Whilst such expression systems can be used to screen up to 10 different members of a library, they are not really suited to screening of larger numbers (greater than 10⁶ members). Other screening systems rely, for example, on direct chemical synthesis of library members. One early method involves the synthesis of peptides on a set of pins or rods, such as described in WO84/03564. A similar method involving peptide synthesis on beads, which forms a peptide library in which each bead is an individual library member, is described in U.S. Pat. No. 4,631,211 and a related method is described in WO92/00091. A significant improvement of the bead-based methods involves tagging each bead with a 15 unique identifier tag, such as an oligonucleotide, so as to facilitate identification of the amino acid sequence of each library member. These improved bead-based methods are described in WO93/06121.

Another chemical synthesis method involves the synthesis of arrays of peptides (or 20 peptidomimetics) on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a receptor) and reactive library members occur is determined, thereby identifying the sequences of the reactive library members on the basis of spatial location. These methods are described in U.S. Pat. No. 5,143,854; WO90/15070, WO92/10092; Fodor et al. (991) Science, 251: 767; Dower and Fodor (1991) Ann. Rep. Med Chem., 26:271.

Other systems for generating libraries of polypeptides or nucleotides involve the use of cell free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/1 1922 (Affymax) use the polysomes to display polypeptides for selection. These and all the foregoing documents also are incorporated herein by reference.

An alternative to the use of phage or other cloned libraries is to use nucleic acid, preferably RNA, derived from the B cells of an animal which has been immunised with the selected target, e.g. an enzyme involved in the catalysis of the production of galactose α-1,3-galactose such as α-1-3 galactosyl transferase. RNA thus obtained represents a natural library of immunoglobulins.

Isolation of V-region and C-region mRNA permits antibody fragments, such as Fab or Fv, to be expressed intracellularly. Briefly, RNA is isolated from the B cells of an immunised animal, for example from the spleen of an immunised mouse or the circulating B cells of a llama, and PCR primers used to amplify V_{H} and V_{L} cDNA selectively from the RNA pool. The V_{H} and V_{L} sequences thus obtained are joined to make scFv antibodies. PCR primer sequences may be based on published V_{H} and V_{L} sequences.

Selection of Intracellular Antibodies

Where antibodies are expressed in the cell cytoplasm (where the redox conditions are unlike those found in the ER) folding and stability problems may occur resulting in low expression levels and the limited half-life of antibody domains. These problems have been attributed to the reducing environment of the cell cytoplasm (Hwang, C., Sinskey, A. J. & Lodish, H. F. (1992) Science 257: 1496-502), which hinders the formation of the intrachain disulphide bond of the V_{H} and V_{L} domains (Biocca, S., Ruberti, F., Tafani, M., Pierandrei-Amaldi, P. & Cattaneo, A. (1995) Biotechnology (NY) 13: 1110-5; Martineau, P., Jones, P. & Winter, G. (1998) J Mol Biol 280: 117-127) important for the stability of the folded protein. However, some scFv have been shown to tolerate the absence of this bond (Proba, K., Honegger, A. & Pluckthun, A. (1997) J Mol Biol 265: 161-72; Proba, K., Worn, A., Honegger, A & Pluckthun, A. (1998) J Mol Biol 275: 245-53) which presumably depends on the particular primary sequence of the antibody variable regions.

In older to identify those immunoglobulins which function intracellularly, immunoglobulins may be tested to identify ability to function intracellularly.

WO00/54057 (Medical Research Council), the disclosure of which is herein incorporated by reference, describes an assay for the identification of such immunoglobulins. Briefly, the assay comprises a system in which a first part of a signal-generating agent is associated with the putative intracellular antibody and a second part of the signal-generating molecule is associated with an intracellular target molecule. A signal is generated by the interaction of the antibody with the target molecule enabling the stable interaction of the two parts of the signal generating molecule. “Stable interaction” may be defined as an interaction which permits functional co-operation of the first and second parts in order to give rise to a detectable result, according to the signaling methods selected for use.

The signal may be any detectable event such as a luminescent, fluorescent or other signal which involves the modulation of the intensity or frequency of emission or absorption of radiation; for example, a FRET signal or the induction of a luciferase gene.

Advantageously, the assay may further comprise a functional assay for the immunoglobulin. For example, where the assay is intended to select immunoglobulins which bind to α-1,3 galactosyl transferase, the immunoglobulins may be tested in an assay to determine any modulating activity on the production of galactose α-1,3 galactose epitope.

Polynucleotide Constructs

In order to provide immunoglobulins within cells of transgenic organisms of the invention, the cells of the transgenic organism are advantageously transfected with a polynucleotide construct which encodes the immunoglobulin.

Polynucleotide constructs encoding an immunoglobulin capable of specifically binding to a retrovirus protein form aspects of the present invention. In addition, polynucleotide constructs encoding an immunoglobulin capable of specifically binding to an enzyme which catalyses the production of galactose α-1,3-galactose and vectors comprising such constructs form aspects of the present invention.

It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

Polynucleotide constructs of the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to educe the in vivo activity or life span of polynucleotides of the invention.

The terms “variant”, “homologue” or “derivative” in relation to the nucleotide sequence of the present invention include any substitution of, variation of, modification of, replacement of, deletion of or addition of one (or more) nucleotides from or to the sequence providing the resultant nucleotide sequence encodes an immunoglobulin which specifically binds an enzyme which catalyses the production of galactose α-1,3-galactose or which encodes an intracellular immunoglobulin able to bind specifically to an endogenous retroviral particle.

Where the polynucleotide of the invention is double-stranded, both strands of the duplex, either individually or in combination, are encompassed by the present invention. Where the polynucleotide is single-stranded, it is to be understood that the complementary sequence of that polynucleotide is also included within the scope of the present invention.

Polynucleotides of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein. Preferred fragments are less than 500, 200, 100, 50 or 20 nucleotides in length.

Polynucleotides such as DNA polynucleotides according to the invention may be 20 produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using PCR (polymerase chain reaction) cloning techniques. This will involve making a pair of primers (e.g. of about 15 to 30 nucleotides) flanking a region of the sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Vectors

Polynucleotides of the invention can be incorporated into a recombinant replicable vector. Such a vector may be any suitable vector, but vectors capable of inserting large amounts of nucleic acid, sufficient to encode an entire immunoglobulin heavy chain locus, are preferred. Such vectors include artificial chromosomes, such as YACs.

The vector may be used to replicate the nucleic acid in a compatible host cell. The construct may be recovered from the host cell. Suitable host cells include bacteria such as E. coli, yeast, mammalian cell lines and other eukaryotic cell lines, for example insect Sf9 cells.

The construct of the present invention may also be incorporated into a vector capable of inserting the construct into a recipient genome and thus achieving transformation.

Preferably, a polynucleotide construct of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under conditions compatible with the control sequences.

The control sequences may be modified, for example by the addition of further transcriptional regulatory elements to make the level of transcription directed by the control sequences more responsive to transcriptional modulators.

Vectors of the invention may be transformed or transfected into a suitable host cell as described below to provide for expression of an immunoglobulin of the invention. This process may comprise culturing a host cell transformed with an expression vector as described above under conditions to provide for expression by the vector of a coding sequence encoding the immunoglobulin, and optionally recovering the expressed immunoglobulin.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term “promoter” is well-known in the art and encompasses nucleic acid regions ranging in size and complexity from minimal promoters to promoters including upstream elements and enhancers.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter, the Rous sarcoma virus (RSV) LTR promoter or the human cytomegalovirus (CMV) IE (Immediate Early) promoter.

It may also be advantageous for the promoters to be inducible so that the levels of expression of the heterologous gene can be regulated during the lifetime of the cell. Inducible means that the levels of expression obtained using the promoter can be regulated. Examples of inducible systems which may be used include the Tet-Off gene expression system (Gossen, M. & Bujard, H. (1992)) the Tet-On system (Gossen, M., et al (1995) Science 268:1766-11769), the tamoxifen inducible system (Indra et al., Nucl Acid Res. 27, 4324-27, 1999) an RU41 8 inducible system Tsujita et al., J. Neuroscience, 19, 10318-23, 1999), and the lac operator—repressor system, which has recently been shown to be functional in mammals, in particular the mouse (Cronin et al, Genes and Development, 15, 1506-1517 (2001)).

In preferred embodiments of the invention, the production of immunoglobulin able to bind specifically to an enzyme which catalyses the production of galactose α-1,3-galactose is maintained throughout the lifetime of the cell in order to inhibit completely the production of galactose α-1,3-galactose. Similarly, in those embodiments in which immunoglobulins able to bind specifically to an endogenous retroviral particle are produced, it is desired to maintain immunoglobulin production throughout the lifetime of the cell in order to prevent release of PERV particles.

In addition, any of these promoters may be modified by the addition of further regulatory sequences, for example enhancer sequences. Tissue-specific enhancers capable of regulating expression in antibody-producing cells are preferred. In particular, the heavy-chain enhancer required for the successful activation of the antibody gene locus in vivo (Serwe, M., and Sablitzky, F., EMBO J. 12, p2321-2321, 1993) may be included. Locus control regions (LCRs), particularly the immunoglobulin LCR, may also be used. Chimeric promoters may also be used comprising sequence elements from two or more different promoters.

In addition to a promoter and the construct, vectors of the present invention preferably contain other elements useful for optimal functioning of the vector in the mammal into which the vector is inserted. These elements are well known to those of ordinary skill in the art, and are described, for example in Sambrook et al., Molecular Cloning: A Laboratory Manual Cold Spring Harbor Laboratory Press, 1989.

Vectors used for transforming mammalian embryos are constructed using methods well known in the art, including, without limitation, the standard techniques of restriction endonuclease digestion, ligation, plasmid and DNA and RNA purification, DNA sequencing, and the like as described, for example in Sambrook, Fritsch, and Maniatis, eds., Molecular Cloning: A Laboratory Manual, (Cold Spring Harbor Laboratory Press, Cold Spring Harbor, N.Y. [1989]). If desired, analysis to confirm correct sequences in the constructed vectors is performed in a known fashion. The presence of a desired construct may be measured in a cell directly, for example, by conventional Southern blotting, dot blotting, PCR or in situ hybridisation, using an appropriately labelled probe which may be based on a sequence present in the gene. Those skilled in the art will readily envisage how these methods may be modified, if desired.

Host Cells

Vectors and polynucleotides of the invention may be introduced into host cells for the purpose of replicating the vectors/polynucleotides and/or expressing the immunoglobulins of the invention encoded by the polynucleotides of the invention. Although the immunoglobulins of the invention may be produced using prokaryotic cells as host cells, it is preferred to use eukaryotic cells, for example yeast, insect or mammalian cells, in particular mammalian cells.

Vectors/polynucleotides of the invention may introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation. These are discussed further below.

Transgenic Animals

The constructs of the present invention may be introduced into an animal to produce a transgenic animal. The invention thus provides a transgenic animal including a construct of the invention.

In a “transgenic animal”, the transgene is contained in essentially all of the animal's cells, including germ cells, such that it can be transmitted to the animal's offspring. In a “chimeric animal”, the transgene is contained in at least some cells of the animal, but germ line transmission is not necessarily possible. The transgene may be limited to particular somatic tissues. In the present invention, “transgenic animals”, in which germ line transmission of the transgene is possible, is preferred. However, the production of a chimeric animal in which the transgene is produced in a particular tissue or organ may be of use in, for example, the provision of that tissue or organ for xenotransplantation. Thus, references to “transgenic animals” in the present application should be understood to include reference to chimeric animals, unless the context demands otherwise.

Techniques for producing transgenic animals, which may be used in the methods of the invention, are well known in the art. A useful general textbook on this subject is Houdebine, Transgenic animas—Generation and Use (Harwood Academic, 1997), which provides an extensive review of the techniques used to generate transgenic animals from fish to mice and cows.

Embryo micromanipulation technologies now permit introduction of heterologous DNA into, for example, fertilised mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In one embodiment, developing embryos are infected with a retrovirus containing the desired DNA, and transgenic animals produced from the infected embryo. In a preferred embodiment, however, the appropriate DNAs are coinjected into the pronucleus of embryos, preferably at the single cell stage, by standard methods. Injected eggs are then transferred, either directly or after culturing, into the oviducts of pseudopregnant recipients and the embryos allowed to develop into mature transgenic animals. Those techniques as well known (see reviews of standard laboratory procedures for microinjection of heterologous DNAs into mammalian fertilised ova, including Hogan et al., Manipulating the Mouse Embryo, (Cold Spring Harbor Press 1986); Krimpenfort et al., Bio/Technology 9:844 (1991); Palmiter et al., Cell, 41: 343 (198S); Kraemer et al., Genetic manipulation of the Mammalian Embryo, (Cold Spring Harbor Laboratory Press 198S); Hammer et al., Nature, 315: 680 (198S); Wagner et al., U.S. Pat. No. 5,175,385; Krimpenfort et al., U.S. Pat. No. 5,175,384, the respective contents of which are incorporated herein by reference).

Transgenic animals may also be produced by nuclear transfer technology as described in Schnieke, A. E. et al., 1997, Science, 278: 2130 and Cibelli, J. B. et al., 1998, Science, 280: 1256. Using this method, fibroblasts from donor animals are stably transfected with a plasmid incorporating the coding sequences for a polypeptide of interest under the control of regulatory sequences. Stable transfectants are then fused to enucleated oocytes, cultured and transferred into female recipients.

By way of a specific example for the construction of transgenic mammals nucleotide constructs comprising a sequence encoding a DNA binding molecule are microinjected using, for example, the technique described in U.S. Pat. No. 4,873,191, into oocytes which are obtained from ovaries freshly removed from the mammal. The oocytes are aspirated from the follicles and allowed to settle before fertilisation with thawed frozen sperm capacitated with heparin and prefractionated by Percoll gradient to isolate the motile fraction.

The fertilised oocytes are centrifuged, for example, for eight minutes at 15,000 g to visualise the pronuclei for injection and then cultured from the zygote to morula or blastocyst stage in oviduct tissue-conditioned medium. This medium is prepared by using luminal tissues scraped from oviducts and diluted in culture medium. The zygotes must be placed in the culture medium within two hours following microinjection.

Oestrous is then synchronized in the intended recipient mammals, by administering coprostanol. Oestrous is produced within two days and the embryos are transferred to the recipient 5-7 days after oestrous. Successful transfer can be evaluated in the offspring by Southern blot.

Alternatively, the desired constructs can be introduced into embryonic stem cells (ES cells) and the cells cultured to ensure modification by the transgene. The modified cells are then injected into the blastula embryonic stage and the blastulas replaced into pseudopregnant hosts. The resulting offspring are chimeric with respect to the ES and host cells, and nonchimeric strains which exclusively comprise the ES progeny can be obtained using conventional cross-breeding. This technique is described, for example, in WO91/10741. Further details of methods of constructing transgenic animals, for example pigs, can be found in U.S. Pat. No. 6,166,288.

Alternative methods for delivery and stable integration of genes encoding immunoglobulins into the genome of host animals include the use of viral vectors, such as adenoviral vectors, retroviral vectors, baculoviral vectors and herpesviral vectors. Such techniques have moreover been described in the art, for example by Zhang et al. (Nucl. Ac. Res., 1998, 26:3687-3693).

Analysis of animals which may contain transgenic sequences may be performed by either PCR, Northern or Southern blot analysis using a probe that is complementary to at least a portion of the transgene following standard methods. Western blot analysis using a ligand specific for the antibody encoded by the transgene may be employed as an alternative or additional method for screening. Typically, the tissues or cells believed to express the transgene at the highest levels are tested, although any tissues or cell types may be used for this analysis.

Progeny of the transgenic mammals may be obtained by mating the transgenic mammal with a suitable partner, or by in vitro fertilisation of eggs and/or sperm obtained from the transgenic mammal. Where in vitro fertilisation is used, the fertilised embryo may be implanted into a surrogate host or incubated in vitro, or both. Where mating is used to produce transgenic progeny, the transgenic mammal may be backcrossed to a parental line. Using either method, the progeny may be evaluated for the presence of the transgene using methods described above, or other appropriate methods.

Uses of the Invention

The methods of the preset invention may be used to provide transgenic animals, on the cell membranes of which expression of the xenoantigen galactose α-1,3 galactose is reduced or eliminated. Cells, organs or tissues from such organisms may therefore be used in xenotransplantation to humans with the risk of hyperacute rejection of the transplant much reduced. As the immunoglobulins inhibiting the production of the xenoantigen are expressed from polynucleotides integrated into the genome of the transplanted tissue, the immunoglobulins may be expressed throughout the lifetime of the tissue, providing constant protection from rejection by the recipient without the need for administration of such immunoglobulins to the recipient of the transplant.

Moreover, the methods of the invention may be used to inhibit the release of endogenous retroviral particles from cells of animals such as pigs by providing transgenic animals that do not release PERVs due to the presence of blocking antibodies and thus reduce the risk of infection of a recipient.

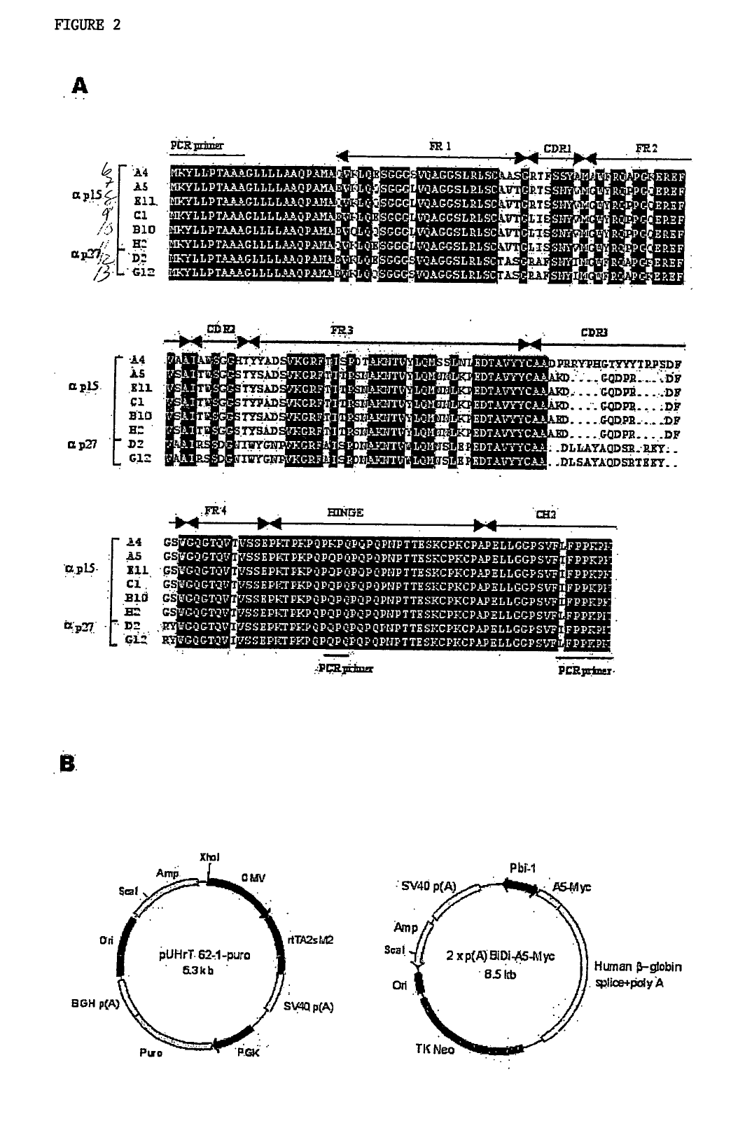

Immunoglobulins which specifically bind to PERV retroviral proteins, such as the gag binders indentified in FIG. 2, may be used in the detection or diagnosis of PERVs.

The invention is further described, for the purposes of illustration only, in the following examples.

### EXAMPLES

Experimental Protocol

Antigen Preparation and Immunisation

PERV-B gag cDNA (AJ13381) was amplified from PK15 cell RNA using the forward primer: Gag fw/Asp (5′ ATAGGTACCATGGGACAGACAGTGACTACC 3′) and reverse primer: Gag rv/Hind (5′ ATAAGCTTGTCCGAACCCCGTCTCCCCTA 3′). The 1.6 kb gag cDNA was cloned into the pET30-a expression vector (Novagen Inc. Winconsin, USA) and overdressed upon IPTG induction in E. coli B121 DE3 (pLysS). p30 was cloned into pTRCB and parts of p15 were cloned into pGEX3x.

Purified 60 kD Gag protein was used for immunisation of a New Zealand rabbit that yielded in a polyclonal rabbit antiserum against the PERV's Gag. The same protein was used for the immunisation of a young adult male Lama glama. The immunisation schedule was as previously described by van der Linden et al. (33)

Immunoelectron Microscopy

PK15 cells were fixed in 4% Paraformaldehyde and prepared for ultracryotom as previously described (34). Ultrathin cryosections (75 nm) were immunolabeled with llama polygonal antiserum against Gag (1:250) followed by the goat anti-llama Ig G antibody (1:250)(Bethyl Laboratories, Inc. Texas, USA) and rabbit anti-goat antibody conjugated with 15 nm colloidal gold particles (1:20) (Aurion, Wagenigen, NL) as described by Geuze et al. (35)

Library Construction and Screening

Total RNA was isolated from peripheral lymphocytes of the immunised llama using Ultraspec RNA isolation system (Biotecx laboratories, Inc, Houston, Tex. USA). After purification of poly A+ RNA (Oligotex 70022, Qiagen), cDNA was made using oligo dT. DNA fragments encoding VHH fragments were amplified by PCR using specific primers: Vh1back SfiI primer (36) in combination with Lam01 NotI primer (5′CAGAAATGGAGCGGCCGCCTTGGGTTTTGGDGGGGAAGAKGAAGACDG ATG-3′) or Lam03 NotI (5′ CCTCGGGGTCGCGGCCGCCACRTCCACCACCACRCAYGTGACCT-3′) from exon CH2 and LH NotI (5′-GGATTGGGTTGCGGCCGCTGGTTGTGGTTGTGGTTGTGGTTTTGGTGTCTG GGGTTC-3′) from the long hinge. The amplified VHHs (˜500 bp) were separated by gel electrophoresis from the VHs of conventional antibodies containing the CH1 exon (˜800 bp) and gel purified. The isolated DNA was SfiI/NotI digested and cloned into SfiI/NotI of the phagemid vector pHEN1 (37).

Transformation into TG1 electrocompetent cells yielded in a llama single chain antibody library with estimated size of 10⁶ recombinants. Two rounds of selection were performed using panning on antigen adsorbed onto plastic (immunotubes coated with 30 μg/ml and 10 μg/ml of purified Gag protein).

BIAcore Measurements

Experiments were carried out on a BIAcore 3000 Surface Plasmon Resonance biosensor. Purified Gag protein was immobilised on a CM5 sensor chip to a level of 600 Resonance Units (RU, arbitrary binding response units) using the standard NHS-EDC kit supplied by the manufacturer. Anti myc-tag antibody was immobilised on a separate flowcell of the same sensorchip to a level of 2400 RU. A flowcell treated with the same procedure but with no protein was used as a control.

The interaction buffer was 20 mM Hepes, pH 7.4, 150 mM NaCl, 2 mM EDTA and 0.005% Tween 20. Dilutions of different periplasmic fractions in the above buffer were firstly injected over the anti-myc spice and adjusted so as to give the same response and thus ensure that the same amount of anti-Gag VHH was present. The adjusted dilutions of periplasmic fractions were then passed at the same time over the control and Gag surfaces and the response at equilibrium was recorded. The amount of non-specific binding was small (less than 5% of the specific) and was subtracted from the specific response. Regeneration of the surfaces was accomplished with a 5ì1 pulse of 0.005% SDS, which resulted in complete dissociation of bound protein and did not affect the binding capacity of the surface for subsequent interactions. Affinity or kinetic constants could not be obtained because of the inability to measure the exact concentration of VHH in the periplasmic fractions and therefore results are presented as relative Gag binding at equilibrium.

Epitope Mapping

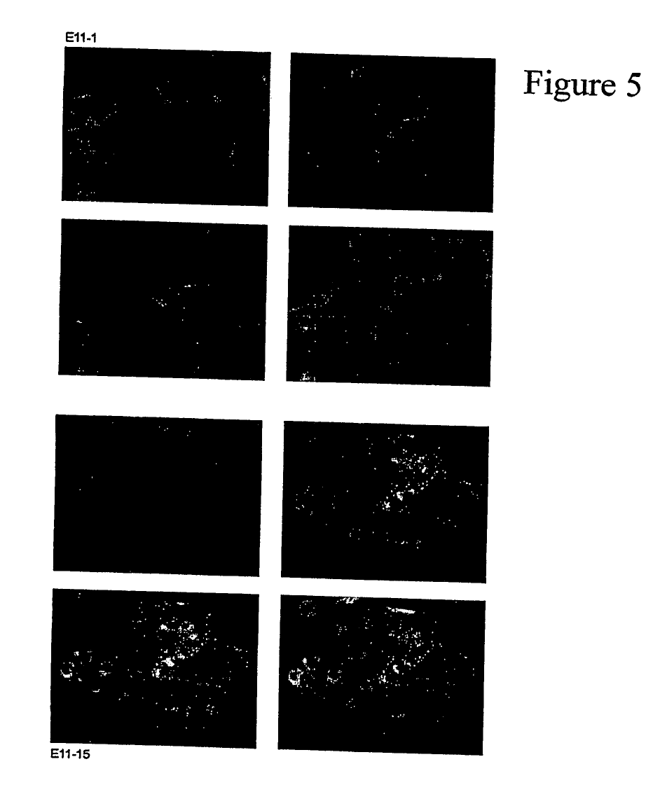

Normal and mutant p15 proteins are expressed using routine methods. Briefly, the DNA encoding the p15 protein (or mutants, as indicated in FIG. 5) is cloned into the bacterial expression vector pET (or pGEX3x) and transfected into E. Coli B121 DE3. Expression is induced and the protein is analysed by Western Blotting using the A5 antibody.

Cell Culture, Transfections and Doxycycline Induction

The PK15 cells were grown in Dulbecco's modified Eagle medium/Ham's F10 (Gibco-BRL, NL) containing 10% foetal calf serum and in later stages supplemented with the appropriate selection markers. The tet-on regulatory plasmid (pUHrT 62-1, generous gift from Dr. Bujard) containing rtTA2^{s} sequence was modified by introduction of the puromycin resistance gene as an eukaryotic selection marker. The resulting plasmid, pUHrT 62-1-puro was ScaI linearised and transfected into PK15 cells using SuperFect Transfection Reagent (Qiagen, Calif., USA) according to the manufacturer instructions. Clones were selected on puromycin 1 μg/ml (Sigma, Zwijndrecht, NL), and screened in a transient transfection assay with the pBI-EGFP-Luc reporter plasmid (Clontech, Calif., USA). Each clone was tested for luciferase and EGFP expression (16) with and without doxycycline induction (500 ng/ml). The clone that gave highest level of luciferase activity and EGFP expression in “on” state and no background in “off” state was used for the transfection experiments with the antibody coding genes.

After transfection with ScaI linearised 2×p(A) BiDi-A5-Myc plasmid, clones were selected and grown in 800 μg/ml G418 (Gibco, UK). Tet on line and clones 13 and 17 were cultured to 40% confluency in 6-well plates with 3 ml of media. Dox was added to half of the wells, all the wells were washed once after 8 hrs of induction to remove residual virus and the medium replaced for another 48 hrs of incubation. The cells were collected and used for Western blot, immunofluorescent staining and immunoelectron microscopy. The supernatant was collected for RTassays or RT-PCR.

RT Assay

C-type Mn²⁺-dependent RT activity assay was performed on the cell-free supernatants from each clone in the un-induced and induced state using Cavidi HSkit (Cavidi Tech Ab, Uppsala, Sweden) according to the manufacturer instructions.

RT-PCR

Culture supernatant was harvested, filtered through 0.45-μm-pore-size filter and virions were pelleted by ultracentrifugation for 2 hrs at 150000×g/30000 rpm. Viral RNA was isolated using a commercially available kit (including a DNase step, Qiagen, Calif., USA). c/DNA was synthesized using oligo dT and super RT (HT Biotechnology, Cambridge, UK). A previously described env specific set of primers for the PCR of PERV-A (p1 206/p1 205) (32), PERV-B (p1 170/p1 171) (1) and PERV-C (p1 172/p1 173) (1) were used. In addition, a pol specific set of primers (for A, B and C type: pol fw 5′ATACTCCCCTGCTACCGGTT 3′ and pol rv 5′CAAGAGGTTATAAGGGTTCGG 3′) was used with the following cycle parameters: 92° C. for 4 min, 36 cycles of 92° C. for 1 min, 53° C. for 1 min, 72° C. for 1 min, followed by 10 ml at 72° C.

Immunofluorescence

Cells were grown on coverslips, fixed in 4% paraformaldehyde and permeabilized with 0.5% triton. Gag was visualised using FITC coupled goat a rabbit IgG (Nordic Immunological Laboratories B.V, NL) as a secondary antibody. VHH expression was detected with Alexa-594 coupled goat α mouse IgG (Molecular Probes, NL). For nuclear staining 4′-6-diamino-2-phenylindole (DAPI, Sigma, Zwijndrecht, NL) was used.

Results

Here, we describe the identification and selection of different llama single chain antibodies in the form of variable heavy chain fragments (VHH) raised against group specific antigen Gag. Importantly we show that intracellular expression of such antibodies inhibits the production of viral particles. They can also be used for detection of PERVs. A number of different anti-sera have already been validated in immunological tests for the detection/diagnosis of PERVs in biological materials (14-17).

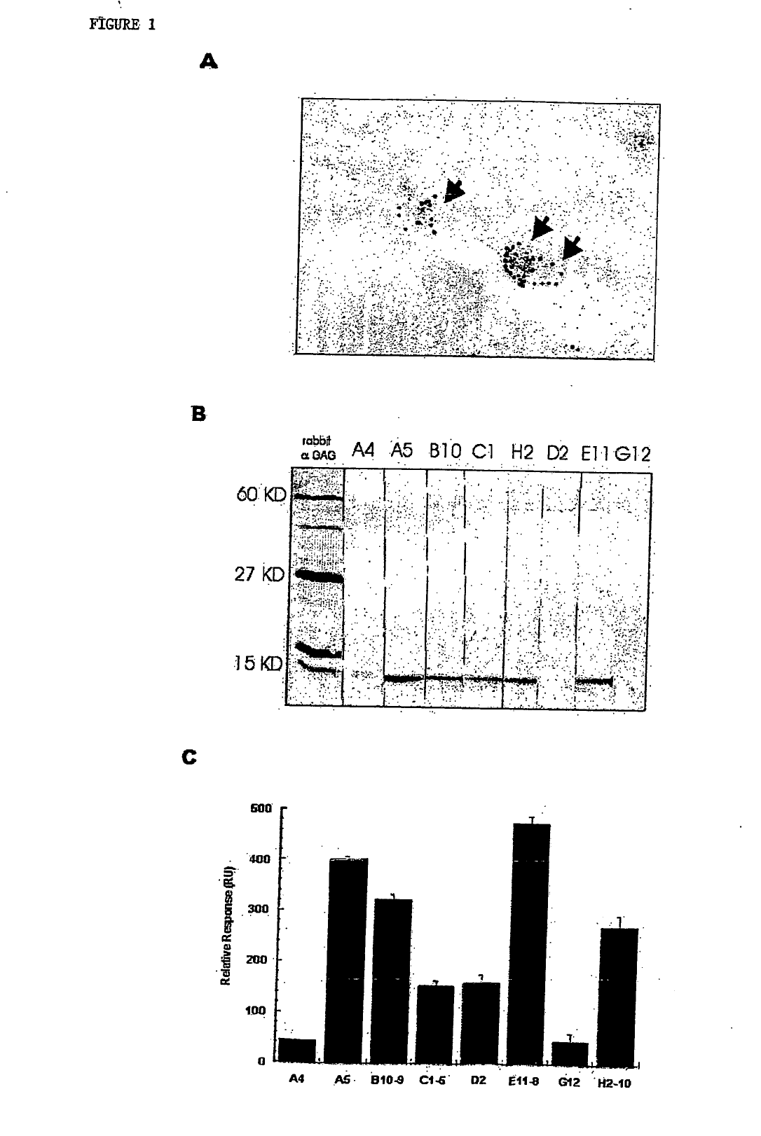

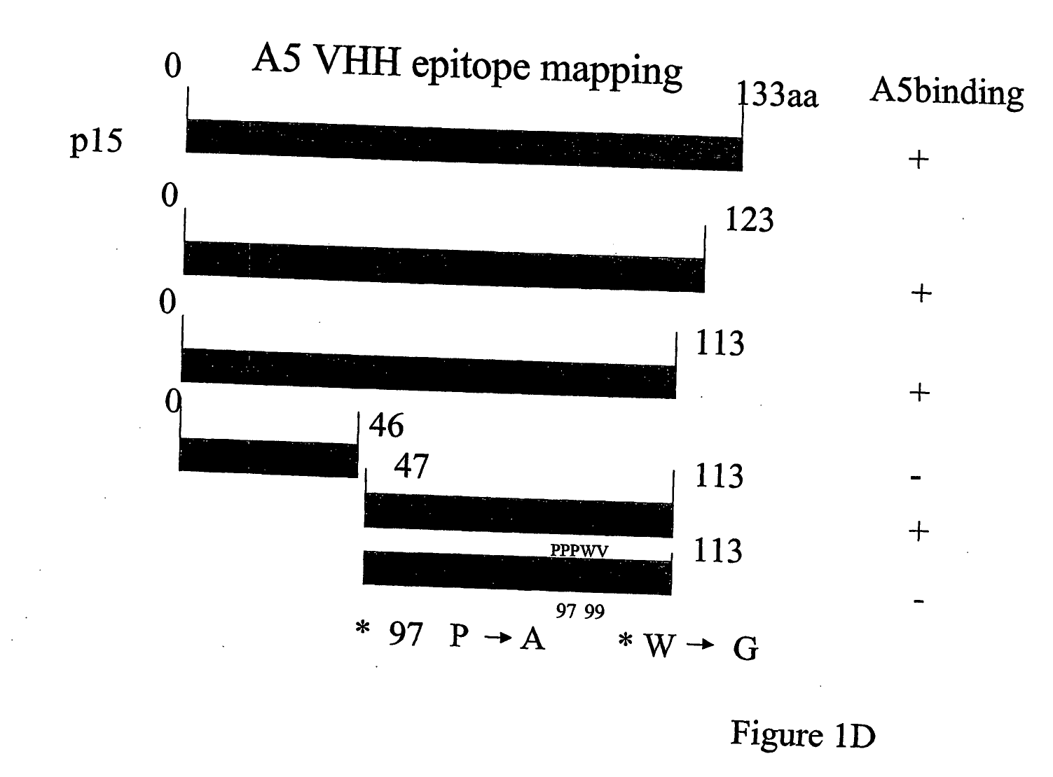

PERV-B gag cDNA was amplified from porcine PK15 cell RNA, expressed in bacteria and the resulting protein was used for immunisation of a New Zealand rabbit that yielded in a polyclonal antiserum against the PERV's Gag. The same protein was used for the immunisation of a young adult male Lama glama. The specificity of both the rabbit and llama polyclonal antisera was tested on Western blots, immunocytochemistry and immunoelectron microscopy. Both sera recognise the precursor 60 KD Gag polyprotein, intermediate forms and further processed matured forms of the viral structural proteins (major capsid (p27), matrix (p15), inner coat and nucleocapsid protein). Immunoelectron microscopy on PK15 cells cryosections, showed specific labelling of the virus particles (FIG. 1A).

In order to isolate the genes coding for the llama antibodies, cDNA was synthesized from RNA isolated from peripheral lymphocytes of the immunised llama and cloned to yield an immune llama single chain antibody phagemid library of 10⁶ clones. Purified Gag protein was used to screen the library. Two rounds of selection were performed using panning on antigen adsorbed onto plastic. The clones positive in an ELISA assay using anti-myc antibody (9E10)(Covane, Calif., USA) for detection of VHHs, were analysed at the DNA level by HinfI fingerprinting.

8 different clones (A4, A5, E11, H2, B10, C1, D2 and G12) were obtained and sequenced (FIG. 2A). They all originated from a long hinge (IgG2) single chain antibody of Lama glama and bind to the Gag protein used for the immunisation as well as to the Gag from PK15 cell lysate and the cell-free viral supernatant. All antibodies were tested against separately expressed cleavage products of Gag polyprotein (data not shown). D2 and G12 bind to p27 (the major capsid protein), while A5, E11, H2, B10 and C1 bind to matrix protein p15 (FIG. 1B). For purification and mass production purposes, all 8 single domain antibodies were shortened by removal of the CH2 region and part of the binge region. This made the his tag accessible for purification purposes (on Ni⁺ beads) and resulted in the production of satisfactory amounts of VHH fragments in the periplasm of bacteria. All 8 periplasmic fractions of the VHHs were shown to be active by BIAcore analyses (18) in which the Gag antigen was immobilised on CM5 sensorchips. The binding affinity was in the order E11>A5>B10>H2>D2>C1>G12>A4 (FIG. 1C).

FIG. 1D shows A5 VHH epitope mapping. The table shows a plus when the A5 antibody detects the full length or mutant protein and a minus when it does not recognise the protein because at least one of the epitopes has been lost. This Figure shows that the epitope of the gag protein p15 (133 amino acids in size) that is recognised by the A5 antibody maps between amino acid 47 and 113 and involves the sequence PPPWV.

One of the highest affinity binders, A5 antibody, was tested for its capacity to block viral production in PK15 cells. For this purpose we used the tetracycline inducible system (tet on) (19). PK15 cells containing the tet-on regulatory construct (FIG. 2B) were stably transfected with the A5 single domain expression vector (FIG. 2B).

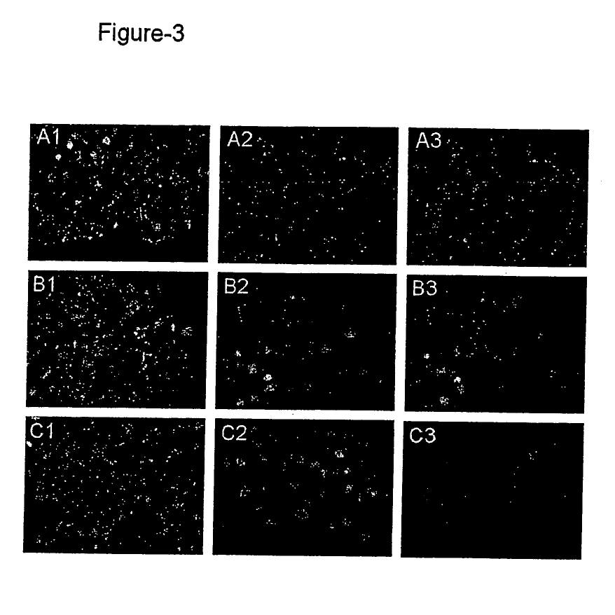

Clones were kept on a double selection using puromycin and G418. After doxycycline (dox) induction, they were screened for expression of the A5 antibody by immunofluorescence using an anti-myc antibody. The expression of antibody differed from clone to clone, ranging from a few percent of cells expressing in some clones, up to a maximum of 90-95% of the cells expressing the A5 VHH. (clone 13). Subcloning or new transfections (also with non tet systems) did not yield any clones where all cells express the antibody, thus we proceeded with two clones, clone 17 (expressing the single domain antibody in ˜30-35% of the cells upon dox induction) and clone 13 (expressing in 90-95% of the cells upon induction). The Gag antigen could be detected by rabbit polyclonal antiserum in all of the non-induced cells (FIG. 3, A1, B1, C1). It is also detectable in those cells that were induced but did not express single chain antibody (FIG. 3, A2-3, B2-3, C2-3). Gag is detected at the plasma membrane in a punctate pattern. However, when the A5 VHH is expressed, the level of Gag protein drops below detection and the punctate staining pattern at the plasma membrane is lost (FIG. 3, B2-3, C2-3).

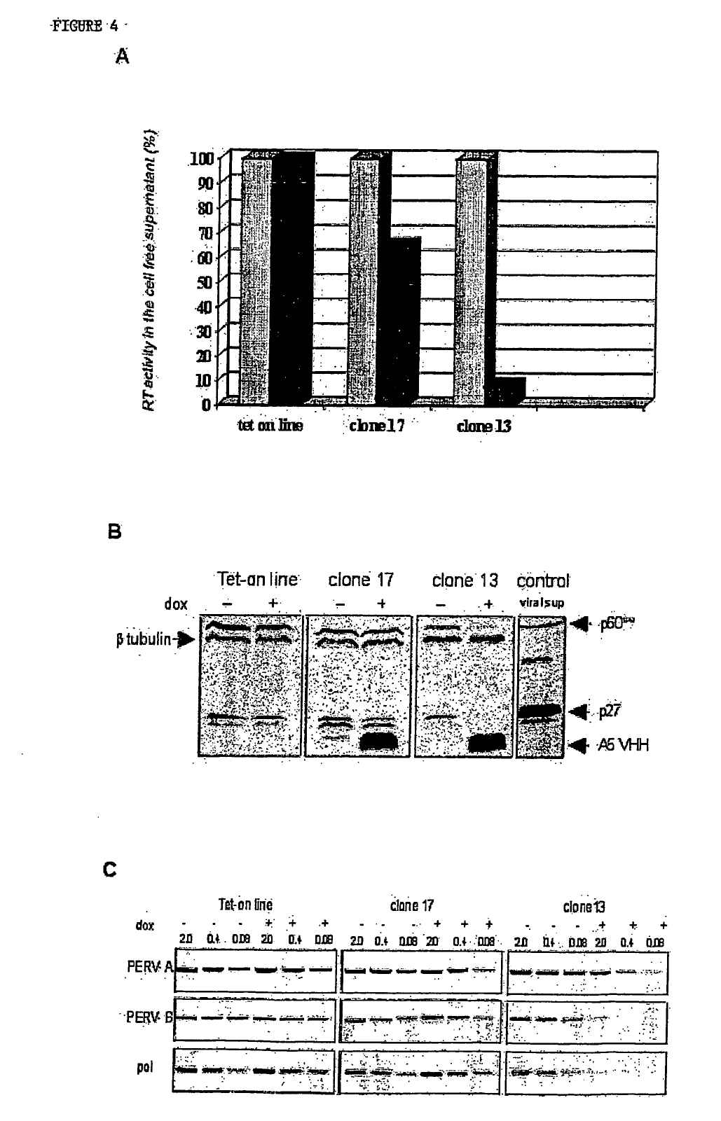

The few cells that do not express the VHH after induction still do show such staining. Occasionally a faint perinuclear staining of Gag can be seen (FIG. 3, B3, C3) in the cells expressing the antibody domain indicating that the Gag protein, that is still produced, rapidly disappears early in the viral assembly process. This is confirmed by Western blot analysis. Cell lysates from tet-on PK15 cell line, not expressing the A5 VHH before and after induction, have the same level of expression of the Gag precursor protein and p27. In the clones that express the A5 upon dox induction, the viral proteins are reduced proportionally to the number of cells expressing the antibody in each clone, using β tubulin expression as a control (FIG. 4B). In both clones (17 and 13), the RT activity remaining in the supernatant collected from dox induced, VHH expressing cells, was greatly reduced in comparison to the supernatant collected from un-induced state of the same clone or control tet-on line regardless of its state of induction. As expected, the largest reduction in RT activity was observed in the clone with the highest percentage of antibody expressing cells. RT activity in this case (clone 13) was reduced to approximately 7% of the activity in un-induced state (FIG. 4A). This residual RT activity correlates very well with the number of cells in the cloned population that do not express the antibody and which still produce viral particles (FIG. 3). It is therefore unlikely that other (as yet unknown) retroviruses present in PK15 cells contribute to the residual RT activity. In order to show that both PERV-A and PERV-B production was blocked, the residual viral RNA in the supernatant was reverse transcribed and amplified with PERV A, B or C specific primers corresponding to the pot and env sequences. As expected, PERV-C particles were not present in PK15 cells (data not shown). Judging by RT-PCR done on serial dilutions of viral cDNA, PERV-A and PERV-B were both present and were both reduced by the same percentage as the number of VHH expressing cells in that clone (FIG. 4C).

FIG. 5 shows loss of virus production in PK15 cells containing the tet-on regulatory construct stably transfected with an E11 single domain expression vector.

Two clones, E11-1 and E-11-15 are shown. Induction of expression of the antibody (red stain) results in a loss of virus production (green speckles), although the gag protein is still visible in the cells (diffuse green staining).

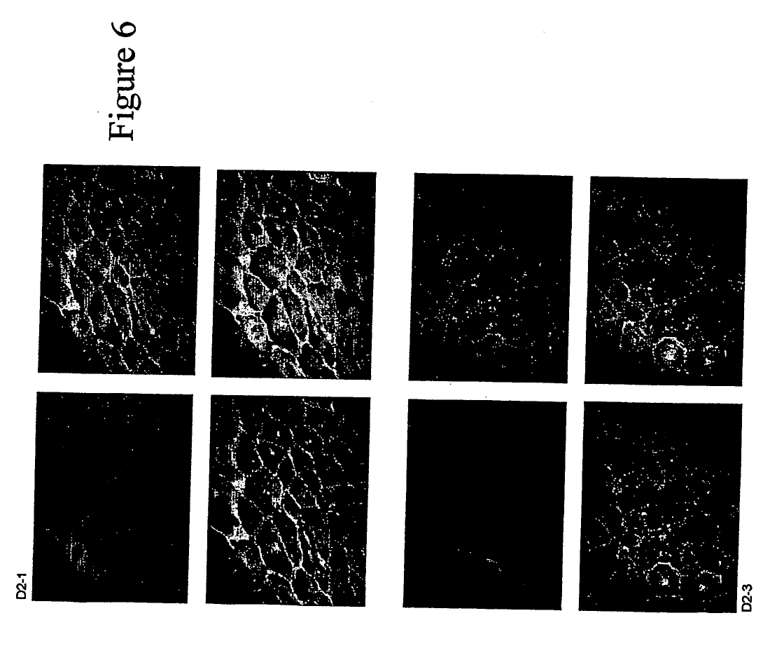

FIG. 6 shows loss of virus production in PK15 cells containing the tet-on regulatory construct stably transfected with D2 single domain expression vector. D2 recognises the p30 gag protein of PERV A and B.

Two clones, D2-1 and D2-2, are shown. Induction of expression of the antibody (red stain) results in a loss of virus production (green speckles), although the gag protein is still visible in the cells (diffuse green staining).

We therefore conclude that our intracellularly expressed antibody against the matrix domain of PERV-B Gag polyprotein completely (>99%) blocks viral production of both PERV-A and B types of retroviruses in PK15 cells. Viral production appears to be blocked early in the process. The antibody interferes with both Gag protein expression and its cleavage products, indicating an inhibition of the maturation process. It also appears to interfere with the transport of Gag to the plasma membrane. In several retroviruses analysed to date, matrix proteins are required for the targeting and interaction of the Gag precursor with the plasma membrane involving N-terminal myristylation and a highly basic domain. They function in envelope glycoprotein incorporation into budding virions and virus particle assembly. They also play additional roles during early and late phases of the viral life cycle (20-24). At present, little work has been carried out to specifically address the function of PERV p15 matrix protein.

Whichever step of the synthesis is blocked, our data show that viral production can be inhibited by single domain VHH, the smallest available intact antigen-binding fragment derived from the functional immunoglobulin, without cytotoxicity.

### REFERENCES

1. Le Tissier, P., Stoy, J. P., Takeuchi, Y., Patience, C. & Weiss R. A. Two sets of human-tropic pig retrovirus. Nature 389, 681-682 (1997).
2. Patience, C., Takeuchi, Y & Weiss, R. A. Infection of human cells by an endogenous retrovirus of pigs. Nature Med. 3, 282-286 (1997).
3. Specke, V., Rubant, S. & Denner, J. Productive infection of human primary cells and cell lines with porcine endogenous retroviruses. Virology 285, 177-180 (2001).
4. Czauderna, F., Fisher, N., Boller, K, Kurth, R & Töjes, R. R. Establishment and characterisation of molecular clones of porcine endogenous retroviruses replicating on human cells. Journal of Virology 74, 4028-4038 (2000).
5. Paradis et al. Search for cross-species transmission of porcine endogenous retrovirus in patients treated with living pig tissue. Science 285, 1236-1241 (1999).
6. Takeuchi, Y. et al. Host range and interference studies of three classes of pig endogenous retrovirus. Journal of Virology 72, 9986-9991 (1998).
7. Dinsmore, J. H., Manhart, C., Raineri, R., Jacoby, D. B.& Moore, A. No evidence for infection of human cells with porcine endogenous retrovirus (PERV) after exposure to porcine fetal neuronal cells. Transplantation 70, 1382-1389 (2000).
8. Cunningham, D. A. et al. Analysis of patients treated with living pig tissue for evidence of infection by porcine endogenous retroviruses. Trends cardiovasc. Med. 11, 190-196 (2001).
9. Herring, C., Cunningham, D. A., Whittam, A. J., Fernandez-Suarez, X. M.& Langford, G. A. Monitoring xenotransplant recipients for infection by PERV. Clinical Biochemistry 34, 23-27 (2001).
10. van der Laan, L. J. et al. Infection by porcine endogenous retrovirus after islet xenotransplantation in SCID mice. Nature 407, 90-94 (2000).
11. Deng, Y. K, Tuch, B. E. & Rowlinson, W. D. Transmission of porcine endogenous retroviruses in severe combined immunodeficient mice xenotransplanted with fetal porcine pancreatic cells. Transplantation 70, 1010-1016 (2000).
12. Denner, J. Immunosuppression by retroviruses: Implications for xenotranspantation. Ann NY Acad. Sc. 862, 75-86 (1998).
13. Nuffield Council on Bioethics. Human-to-animal transplants. The ethics of transplantation. London: Nuffield Council on Bioethics, 1996.
14. Krach, U., Fischer, N., Czauderna, F., Kurth, R., Tönjes, R. R. Generation and testing of a highly specific anti-serum directed against porcine endogenous retrovirus nucleocapsid. Transplantation 7, 221-229 (2000).
15. Herring, C. et al. Monitoring xenotransplant recipients for infection by PERV. Clinical Biochemistry 34, 23-27 (2001).
16. Galbraith, D. N. et al. Design and validation of immunological tests for the detection of porcine endogenous retrovirus in biological materials. Journal of Virological Methods 90, 115-124 (2000).
17. Stephan, O. et al. Porcine endogenous retroviruses (PERVs): Generation of specific antibodies, development of an immunoperoxidase assay (IPA) and inhibition by AZT. Xenotransplantation 8, 310-316 (2001).
18. Kazemier, B., de Haard, H., Boender, P., van Gemen, B. & Hoogenboom, H. Determination of active single chain antibody concentrations in crude periplasmic fractions. J. Immunol. Methods 194, 201-209 (1996).
19. Urlinger, S. et al. Exploring the sequence space for tetracycline-dependent transcriptional activators: Novel mutations yield expanded range and sensitivity. Proc. Natl. Acad. Sci. USA. 97, 7963-7968 (2000).
20. Freed, E. O. HIV-1 Gag proteins: Diverse functions in the virus life cycle. Virology 251, 1-15 (1998).
21. Ono, A., Orenstein, J. M. & Freed E. O. Role of the gag matrix domain in targeting human immunodeficiency virus type I assembly. Journal of Virology 74, 2855-2866 (2000).
22. Rein, A et al. Myristylation site in Pr65 is essential for virus particle formation by Moloney murine leukemia virus. Proc Natl Acad Sci USA 19, 7246-7250 (1986).
23. Yu, X et al. The matrix protein of human immunodeficiency virus type I is required for incorporation of viral envelope protein into mature virions. Journal of Virology 66, 4966-4971 (1992).
24. Levin, R et al. Inhibition of early and late events of HIV-1 replication cycle by cytoplasmatic Fab intrabodies against the matrix protein, p17. Molecular Medicine 3 (2), 96-110 (1997).
25. Dai, Y. at al. Targeted disruption of the α 1,3-galactosyltransferase gene in cloned pigs. Nat. Biotechnology 20, 251-255. (2002).
26. Lai, L. et al. Production of α 1,3-galactosyltransferase knockout pigs by nuclear transfer cloning. Science 295, 1089-1092. (2002).
27. Butler, D. Xenotransplant experts express caution over knockout piglets. Nature 415, 103-104 (2002).
28. Platt, J. L. Knocking out xenograft rejection. Nature biotechnology 20 (3), 231-232 (2002).
29. Cozzi, E. et al. Characterization of pigs transgenic for human decayaccelerating factor. Transplantation 64, 1383-1392 (1997).
30. Cozzi, E. White, D. J. G. The generation of transgenic pigs as potential organ donors for humans. Nature Med. 1, 964-966 (1995).
31. Rother, R. P. et al. A novel mechanism of retrovirus inactivation in human serum mediated by anti-alpha-galactosyl natural antibody. Journal of Experimental Medicine 182, 1345-1355 (1995).
32. Weiss, R. A. Transgenic pigs and virus adaptation. Nature 391, 327-328 (1998).
33. van der Linden, R. et al. Induction of immune responses and molecular cloning of the heavy chain antibody repertoire of Lama glama. Journal of Immunological Methods. 240, 185-195. (2000).
34. Tokuyasu, K. T. A study of positive staining of ultrathin frozen sections. J Utrastruct Res. 63, 287-307 (1978).
35. Geuze H. J., Slot 3. W., van der Ley P. A. & Scheffer R. C. Use of colloidal gold particles in double-labeling immunoelectron microscopy of ultrathin frozen tissue sections. J Cell Biol. 89,653-65 (1981).
36. Orlandi, R., Gussov, D. H., Jones, P. T. & Winter G. Cloning immunoglobulin variable domains for expression by the polymerase chain reaction. Proc. Natl. Acad. Sci. USA 86, 3833-3837. (1989).
37. Hoogenboom, H. R. et al. Multi-subunit proteins on the surface of filamentous phage: methodologies for displaying antibody (Fab) heavy and light chains. Nucleic Acids Research 19, 4133-4137. (1991).

All publications mentioned in the above specification are herein incorporated by reference. Various modifications and variations of the described methods and system of the invention will be apparent to those skilled in the art without departing from the scope and spirit of the invention. Although the invention has been described in connection with sc preferred embodiments, it should be understood that the invention as claimed should not be unduly limited to such specific embodiments.

Indeed, various modifications of the described modes for carrying out the invention which are obvious to those skilled in molecular biology, immunology or related fields are intended to be within the scope of the following claims.

1. A non-human transgenic organism comprising a polynucleotide construct encoding an intracellular immunoglobulin.

2. A non-human transgenic organism of claim 1, wherein the intracellular immunoglobulin binds specifically to an enzyme which catalyses the production of galactose oc-1,3-galactose.

3. The non-human transgenic organism of claim 2, wherein said enzyme is α-1,3 galactosyl transferase.

4. A non-human transgenic organism comprising a polynucleotide construct encoding an intracellular immunoglobulin which binds specifically to a retroviral protein.

5. The non-human transgenic organism of claim 4, wherein said retroviral particle is a porcine endogenous retroviral (PERV) particle protein.

6. The non-human transgenic organism of claim 5, wherein said retroviral particle is the gag protein of a PERV.

7. The non-human transgenic organism of claim 6, wherein said retroviral particle is the p15 matrix domain protein of the PERV gag protein.

8. The non-human transgenic organism of claim 6, wherein said retroviral particle is the p30 capsid protein of the PERV gag protein.

9. The non-human transgenic organism of claim 1, wherein said intracellular immunoglobulin is a single chain antibody.

10. The non-human transgenic organism of claim 9, wherein said single chain antibody is a camelid antibody.

11. The non-human transgenic organism according to any one of the preceding claims wherein said single chain antibody is a llama single domain antibody (VHH) having an amino acid sequence of anyone of the sequences set out in FIG. 2.

12. A method of producing a non-human transgenic organism, said method comprising inserting into the genome of said organism a polynucleotide construct encoding an immunoglobulin which binds specifically to an enzyme which catalyses the production of galactose α-1,3-galactose.

13. The method of claim 12, wherein said enzyme is α-1,3 galactosyl transferase.

14. A method of producing a non-human transgenic organism, said method comprising inserting into the genome of said organism a polynucleotide construct encoding an intracellular immunoglobulin which binds specifically to a retroviral protein.

15. The method of claim 13, wherein said retroviral protein is a porcine endogenous retroviral (PERV) particle protein.

16. The method of claim 12, wherein said intracellular immunoglobulin is a single chain antibody.

17. The method of claim 16, wherein said single chain antibody is a camelid antibody.

18. A method of claim 12, wherein said polynucleotide construct further includes a nucleic acid sequence encoding at least one complement inhibiting protein.

19. The method of claim 18, wherein said complement inhibiting protein is selected from the group comprising CD59, DAF and MOP.

20. A method for preparing organs, tissues or cells for xenotransplantation comprising:
providing a transgenic organism of claim 1; and
isolating said organ, tissue or cell from said transgenic organism.

21. A polynucleotide construct encoding an intracellular immunoglobulin which binds specifically to an enzyme which catalyses the production of galactose α-1,3-galactose.

22. The polynucleotide construct of claim 21, wherein said enzyme is α-1,3 galactosyl transferase.

23. A polynucleotide construct encoding an intracellular immunoglobulin which binds specifically to a retroviral protein.

24. The polynucleotide construct of claim 23, wherein said retroviral protein is a porcine endogenous retroviral (PERV) particle protein.

25. The polynuclotide construct of claim 21, wherein said intracellular immunoglobulin is a single chain antibody.

26. The polynuclotide construct of claim 25, wherein said single chain antibody is a camelid antibody.

27. The polynuclotide construct of claim 21, wherein said construct further comprises a nucleic acid sequence encoding at least one complement inhibiting protein.

28. The polynucleotide construct of claim 27, wherein said complement inhibiting protein is CD59, DAF or MCP.

29. A polynucleotide encoding a gag binding protein having an amino acid sequence as set out in FIG. 2.

30. A vector comprising a polynucleotide construct of claim 21.

31. An immunoglobulin encoded by the polynucleotide construct of claim 21.

32. The immunoglobulin of claim 31, wherein said immunoglobulin binds specifically to an enzyme which catalyses the production of galactose α1,3-galactose.

33. The immunoglobulin of claim 31, wherein said immunoglobulin binds specifically to a retroviral protein.

34. An immunoglobulin having an amino acid sequence of any of the polypeptides set out in FIG. 2.

35. A host cell comprising the nucleic acid sequence of claim 21.

36. A host cell comprising the immunoglobulin of claim 31.

37. A host cell comprising the immunoglobulin of claim 34.


SEQUENCE LISTING (15 sequences)

SEQ ID NO: 1 - DNA, 30 bp, Artificial sequence
  PCR primer Gag fw/Asp
ataggtaccatgggacagacagtgactacc

SEQ ID NO: 2 - DNA, 29 bp, Artificial sequence
  PCR primer Gag rv/Hind
ataagcttgtccgaaccccgtctccccta

SEQ ID NO: 3 - DNA, 51 bp, Artificial sequence
  PCR primer Vh1 back SfiI
cagaaatggagcggccgccttgggttttggdggggaagakgaagacdgatg

SEQ ID NO: 4 - DNA, 44 bp, Artificial sequence
  PCR primer
cctcggggtcgcggccgccacrtccaccaccacrcaygtgacct

SEQ ID NO: 5 - DNA, 57 bp, Artificial sequence
  PCR primer
ggattgggttgcggccgctggttgtggttgtggttgtggttttggtgtctggggttc

SEQ ID NO: 6 - DNA, 20 bp, Artificial sequence
  PCR primer pol fw
atactcccctgctaccggtt

SEQ ID NO: 7 - DNA, 21 bp, Artificial sequence
  PCR primer pol rv
caagaggttataagggttcgg

SEQ ID NO: 8 - protein, 194 aa, Lama glama
MKYLLPTAAAGLLLLAAQPAMAQVKLQESGGGSVQAGGSLRLSCAASGRTFSSYAMAWFRQAPGKEREFVAAIAWSGGHTYYADSVKGRFTISPDTAKNTVYLQMSSLNLEDTAVYYCAADPRRYPHGTYYYTRPSDFGSWGQGTQVTVSSEPKTPKPQPQPQPNPTTESKCPKCPAPELLGGPSVFLFPPKPK

SEQ ID NO: 9 - protein, 190 aa, Lama glama
MKYLLPTAAAGLLLLAAQPAMAEVKLQQSGGGLVQAGGSLRLSCAVTGRTSSNYVMGWYRQPPGQEREFVSAITWSGGSTYSADSVKGRFTITPSNAKNTVYLQMNNLKPEDTAVYYCAAAKDGQDPRDFGSWGQGTQVTVSSEPKTPKPQPQPQPQPQPNPTTESKCPKCPAPELLGGPSVFIFPPKPK

SEQ ID NO: 10 - protein, 190 aa, Lama glama
MKYLLPTAAAGLLLLAAQPAMAQVKLQQSGGGLVQAGGSLRLSCAVTGRTSSNYVMGWYRQPPGQEREFVSAITWSGGSTYSADSVKGRFTITPSNAKNTVYLQMNNLKPEDTAVYYCAAAKDGQDPRDFGSWGQGTQVTVSSEPKTPKPQPQPQPQPQPNPTTESKCPKCPAPELLGGPSVFIFPPKPK

SEQ ID NO: 11 - protein, 190 aa, Lama glama
MKYLLPTAAAGLLLLAAQPAMAEVKLQESGGGLVQAGGSLRLSCAVTGLISSNYVMGWYRQPPGQEREFVSAITWSGGSTYPADSVKGRFTITPSNAKNTVYLQMNNLKPEDTAVYYCAAAKDGQDPRDFGSWGQGTQVTVSSEPKTPKPQPQPQPQPQPNPTTESKCPKCPAPELLGGPSVFIFPPKPK

SEQ ID NO: 12 - protein, 190 aa, Lama glama
MKYLLPTAAAGLLLLAAQPAMAEVQLQQSGGGLVQAGGSLRLSCAVTGLISSNYVMGWYPQPPGQEREFVSAITWSGGSTYSADSVKGRFTITPSNAKNTVYLQMNNLKPEDTAVYYCAAAEDGQDPRDFGSWGQGTQVTVSSEPKTPKPQPQPQPQPQPNPTTESKCPKCPAPELLGGPSVFIFPPKPK

SEQ ID NO: 13 - protein, 190 aa, Lama glama
MKYLLPTAAAGLLLLAAQPAMAQVKLQESGGGLVQAGGSLRLSCAVTGLISSNYVMGWYPQPPGQEREFVSAITWSGGSTYSADSVKGRFTITPSNAKNTVYLQMNNLKPEDTAVYYCAAAEDGQDPRDFGSWGQGTQVTVSSEPKTPKPQPQPQPQPQPNPTTESKCPKCPAPELLGGPSVFLFPPKPK

SEQ ID NO: 14 - protein, 193 aa, Lama glama
MKYLLPTAAAGLLLLAAQPAMAEVKLQESGGGSVQAGGSLRLSCTASGRAFSNYIMGWFRQAPGKEREFVAAIRSSDGNIWYGNPVKGRFAISPQNAKNTVWLQMNNSEPEDTAVYYCAADLLAYAQDSRREYRYWGQGTQVIVSSEPKTPKPQPQPQPQPQPNPTTESKCPKCPAPELLGGPSVFIFPPKPK

SEQ ID NO: 15 - protein, 195 aa, Lama glama
MKYLLPTAAAGLLLLAAQPAMAEVKLQQSGGGSVQAGGSLRLSCTASGRAFSNYIMGWFRQAPGKEREFVAAIRSSDGNIWYGNPVKGRFAISPDNAKNTVWLQMNNSLEPEDTAVYYCAADLSAYAQDSRTEEYRYWGQGTQVIVSSEPKTPKPQPQPQPQPQPNPTTESKCPKCPAPELLGGPSVFLFPPKPK